(19) United States
(12) Patent Application Publication
(10) Pub. No.: US 2006/0189991 A1
(43) Pub. Date: Aug. 24, 2006

(54) Graft anchor

(76) Inventor: Barry T. Bickley, North Andover, MA (US)

Correspondence Address: DALY, CROWLEY, MOFFORD & DURKEE, LLP, SUITE 301A, 354A TURNPIKE STREET, CANTON, MA, 02021-2714, US

(21) Appl. No.: 11/330,900

(22) Filed: Jan. 11, 2006

Related U.S. Application Data
(60) Provisional application No. 60/642,889, filed on Jan. 11, 2005.

(51) Int. Cl. A61B 17/58

(52) U.S. Cl. 606/72

(57) ABSTRACT

A graft anchor includes an interference screw having a passage provided therein and a cross member adapted to be disposed in the passage of the interference screw to secure a graft.

### CROSS-REFERENCE TO RELATED PATENT APPLICATIONS

This patent application claims the benefit of U.S. provisional application No. 60/642,889 filed on Jan. 11, 2006 under 35 U.S.C. §119(e).

### GOVERNMENT RIGHTS

Not applicable.

### TECHNICAL FIELD

The invention generally relates to medical devices and more particularly to methods and apparatus for securing tendons.

### BACKGROUND OF THE INVENTION

As is known in the art, injuries to the anterior cruciate ligament (ACL) of the knee are common in athletically active individuals. Graft fixation on the tibial side remains a source of difficulty and surgical failures. The limiting of tension loss and laxity depends upon fixation and bone quality.

As is known, the anterior cruciate ligament (ACL) is commonly injured in athletically active individuals. Such an injury frequently results in knee instability that requires surgical reconstruction of the ACL in order to return to athletic activity and in many cases to even return to normal daily activities. Two common grafts used are obtained either from the central third of the patellar tendon with a plug of bone from both the patella and the tibial tubercle or from the hamstring tendons.

The patellar tendon grafts are most often fixed in the bone tunnels by interference screws. The interference screw wedges itself between the bone plug on the graft and the wall of the bone tunnel. This technique is familiar to all surgeons who perform this type of surgery and is easy to accomplish. Failure of this technique can still result from slippage of the graft past the interference screw. In pullout studies, this is a frequent failure mode of this type of fixation.

Recently, soft tissue grafts, such as hamstring and other allograft tendons have been gaining in popularity. These graft options have appeal because of the diminished morbidity to patients. Hamstring and soft tissue grafts have had some difficulty in regard to fixation of the graft to the bone, in particular on the tibial side of the knee.

On the tibial side, the ends of the ligament graft need to be fixed and this is often accomplished using an interference screw placed in line with a bone tunnel in which the ligament graft is disposed. The interference screw compresses the graft ligament (e.g. the tendon) against the wall of the bone tunnel. The problem lies in the fact that the bone where the tunnel is made is often soft in this portion of the tibia. This can compromise any method of fixation that relies on compression against this weak bone. Also the line of force that can loosen the graft is directly in line with the tunnel. This can allow the graft to slip beneath the interference screw. Because of this, surgeons will frequently add a second screw and a washer that are placed transversely in the tibia just beyond the far end of the tibial tunnel. The graft ends are then wrapped around the screw. The screw is advanced and the washer captures the tendon beneath it and compresses it against the outside of the bone. While this does provide additional fixation, the line of force for loosening of the graft is once again in line with the graft and can pull the tendon from beneath the washer.

### SUMMARY OF THE INVENTION

In accordance with the present invention a graft anchor includes an interference screw having a passage provided therein and a cross member adapted to be disposed in the passage of the interference screw. In some embodiments, it is also desirable to dispose a washer between a surface of the interference screw and a surface of the cross member such that the cross member exerts a force on the washer to hold the washer against a face of the interference screw.

With this particular arrangement, an easy-to-use graft anchor which provides a large amount of graft holding power is provided. By using both an interference screw and a cross member disposed in the interference screw, two approaches for securing grafts are combined into a single device thereby providing a single device having a graft holding power which is increased compared with the holding power of prior art devices. The device can be used with all standard grafts commonly used for ACL knee reconstruction as well as with grafts used in other types of reconstructions or other applications.

The interference screw is cannulated to follow a pre-placed guide wire. In one embodiment, the screw is not fully threaded on its outer surface but rather has a non-threaded portion in a head region of the screw. This head region can be slightly flared in diameter for the purpose of taking up a greater volume within a hole provided in an outer cortex of the bone which accepts the interference screw. The end of the head is preferably provided having an angle selected to such that the screw head is less prominent from a surface of a bone into which the interference screw is inserted. When the screw is placed obliquely into the bone as is commonly done in ligament reconstruction surgery, head angle is approximately forty-five degrees. A portion of the head can be beveled to accommodate grafts (e.g. tendons or other types of grafts) as they are drawn over the angled face of the head and secured. This bevel can be further grooved longitudinally to facilitate grouping of ends of the grafts together and preventing migration of the grafts during placement of a washer. The interference screw accommodates a cross member which secures the grafts. In one embodiment, the cross member is provided as a screw and the interference screw includes a threaded screw hole which accepts the cross screw. In one embodiment, a central longitudinal axis of the screw hole is provided at an angle which is perpendicular to the angled face.

In one embodiment, the cross member secures a graft in a desired location. In another embodiment, the cross member is in conjunction with a washer. The washer can be provided having a circular, elliptical or any other geometric shape. In one embodiment, the cross member secures the washer and the washer compresses the grafts (e.g. tendon(s)) that are placed over the angled face of the interference screw. The washer can also be provided having a complex shape that helps to minimize prominence above a surface of a bone in which the interference screw is disposed. For example, the washer may be provided having rounded edges and a generally rounded external side while maintaining a generally flat internal side that is disposed against the angled-face of the interference screw.

It should be appreciated that in some embodiments the washer may be not be required while in other embodiments the washer may be provided as an integral part of the cross-member (e.g. the screw may be integrally formed as part of the cross member such that the cross member and washer comprise a single piece). In still other embodiments, the cross member and washer may be provided as individual pieces.

Both the angled face of the interference screw and the under surface of the washer may be textured or grooved. This facilitates keeping the graft ends gathered in place once the graft ends are arranged between the angled face of the interference screw and a surface of the cross-member or washer (in the case where a washer is used to help secure the grafts in place). The textured or grooved surfaces also increase frictional resistance to forces which may pull the graft ends from beneath the cross member (or washer).

To utilize the graft anchor, the interference screw portion is placed along side strands of tendon in a pre-drilled hole in a bone (e.g. a tibial bone tunnel). The interference screw is advanced into the hole until the head is not prominent (e.g. the head end of the interference screw is preferably substantially flush with or even below a surface of the bone into which the interference screw is being inserted). The tendon strands exiting the bone can be drawn over the angled face of the interference screw head. The cross member is disposed into the corresponding hole in the interference screw to secure the grafts. In the case when a washer is used, once the graft strands exiting the bone are drawn over the angled face of the interference screw, the washer is placed across the angled face to thus secure the grafts between the surface of the angled face of the head of the interference screw and a surface of the washer. The cross member is then advanced in to a hole in the interference screw to advance the washer and compress the grafts between the angled face of the interference screw and the surface of the washer.

### BRIEF DESCRIPTION OF THE DRAWINGS

FIG. 1 is a side view of a graft anchor;

FIG. 2 is an isometric view of an interference screw;

FIG. 3 is a side view of an interference screw;

FIG. 3A is a cross-sectional side view of the interference screw of FIG. 3;

FIG. 4 is a side view of an interference screw;

FIG. 4A is a cross-sectional side view of the interference screw of FIG. 4;

FIG. 5 is a side view of a cross screw;

FIG. 6 is a bottom view of a washer;

FIG. 6A is a top view of the washer of FIG. 6;

FIG. 7 is a side view of a graft anchor being inserted into a tibia; and

FIG. 7A is a side view of the graft anchor of FIG. 7 after insertion of the graft anchor in the tibia.

### DETAILED DESCRIPTION

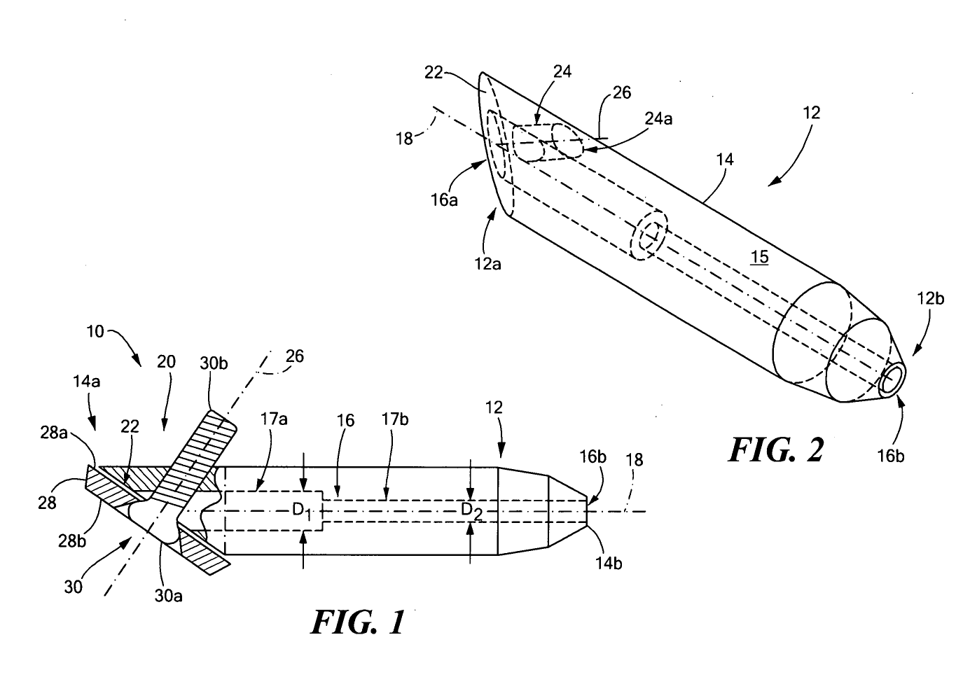

Before describing exemplary embodiments of graft anchors and elements thereof as shown in FIGS. 1-6, it should be appreciated that description herein below is of exemplary embodiments only and is not intended to limit the scope, applicability or configuration of the claimed invention in any way. Rather, the following description provides a convenient illustration for implementing exemplary embodiments of the invention. Various changes to the described embodiments may be made in the function and arrangement of the elements described without departing from the spirit and scope of the invention as set forth in the appended claims.

It should also be appreciated that reference is made herein to “graft ligaments” or more simply “grafts” and that for the purposes of the present invention it should be understood that reference herein to “grafts” includes but is not limited to a ligament or tendon which is harvested from elsewhere in a patient (or from outside the patient) as well as any synthetic devices.

Referring now to FIGS. 1 and 2 in which like reference elements are provided having like designations, a graft anchor 10 includes an interference screw 12, having a bullet shaped body or sidewall 14 having an outer surface 15 and a first opening or passage 16 extending from a first or proximal end 14a of the body 14 to a second or distal end 14b of the body 14. The opening 16 is provided along a central longitudinal axis 18 of the body 14. A first portion 17a of the passage 16 is provided having a first diameter D1 and a second portion 17b of the passage 16 is provided having a second different diameter. A portion 20 of the first end 14a of body 14 corresponds to a so-called “head” of the interference screw 12 and is provided having a surface 22 onto which a washer 28 is disposed and secured with a cross member 30 as will be described below. Both the angled face of the interference screw and the under surface of the washer may be provided having grooves, notches, or other voids provided therein (collectively referred to as a textured surface). In the embodiment of FIGS. 1 and 2, surface 22 is provided at an angle of approximately forty-five degrees with respect to the axis 18.

The interference screw 12 is also provided, having a second opening or passage 24 (FIG. 2) provided therein. The second passage 24 has a first end 24a which opens into at least a portion of the first passage 16 and a second end 24b which extends into at least a portion of the sidewall 14. In the exemplary embodiment shown in FIGS. 1 and 2, the second end 24b of the second passage 24 extends through the sidewall 14. Thus in this embodiment, the first end 24a of the passage 24 terminates in an aperture in a surface of sidewall 14 which defines the opening 16 and the second end 24b of the passage 24 terminates in an aperture in a surface of sidewall 14 which defines the outer surface 15 of the body 14. Also in the embodiment shown in FIGS. 1 and 2, a central longitudinal axis 26 (FIG. 2) of the second passage 24 is provided at an angle which is perpendicular to the surface 22.

The washer 28 has a first or bottom surface 28a and a second or top surface 28b. The washer is disposed over the angled face 22 of the insertion screw 12 such that the bottom surface 28a contacts the surface of the angled face 22. The washer is provided having a hole therethrough. When the washer 28 is disposed on the surface 22, the hole in the washer 28 is aligned with the second passage 24 in the interference screw 12.

A cross member 30, having a first or head end 30a and a second end 30b is disposed through the hole in the washer 28 and into the second passage 24 in the interference screw 12. In one exemplary embodiment, the cross member is provided as a cross screw and the second passage is threaded so as to accept mating threads on the cross screw. In this case, the cross screw is advanced into the corresponding threaded hole 24 to secure the bottom surface 28b washer of the washer 28 against the surface of the angled face 22 of the interference screw 12.

It should be understood that although the embodiment of the interference screw 12 shown and described in FIGS. 1 and 2 does not include threads on the outer surface of the body 14, as will become apparent from the descriptions herein below, other embodiments of the interference screw do include threads on the outer surface of the body 14.

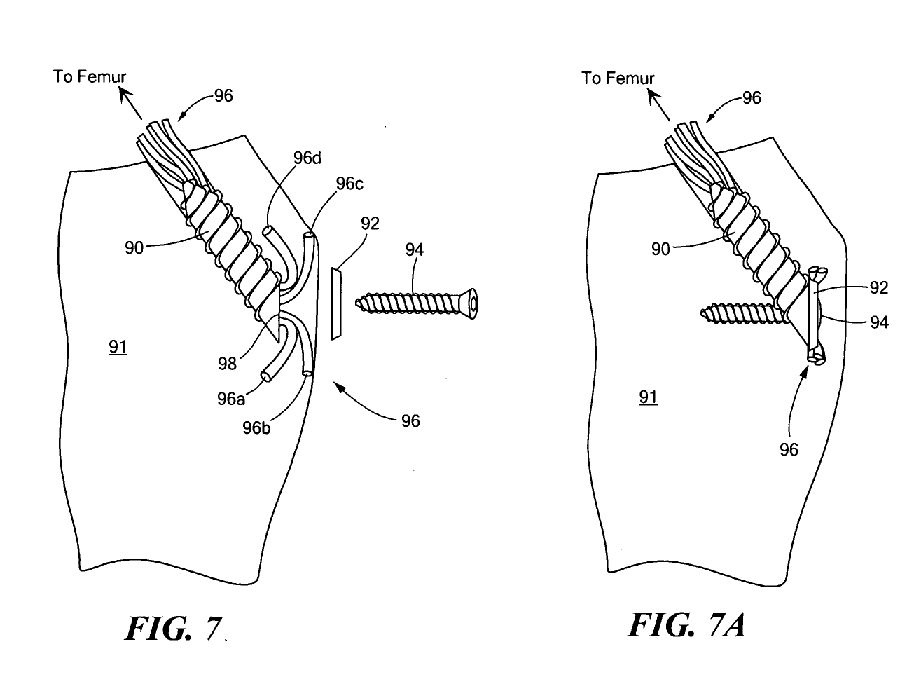

It should also be appreciated that the shaft or body portion of the interference screw 12 may be tapered. That is, the diameter of the shaft may get larger toward the head of the screw. It should also be understood that the shaft may be provided having any length depending upon the nature and type of bone in which it is intended to be used or the purpose for which it is being used. Also, the head end of the interference screw may be flared which results in a tighter fit of the interference screw in a bone tunnel such as a tibia tunnel described below in conjunction with FIGS. 7 and 7A.

The distal guiding tip 14b of the body 14 may be tapered to a point or, as in the illustrated embodiment, may terminate in a beveled tip 14b for pre-tapped applications that employ conventional pre-tapping methodologies (e.g. pre-drilling and the like). It should be appreciated, however, that self-tapping or self-drilling end configurations may be used; for example, tapping flutes or the like. Distal guiding tip 14b serves as a guiding or alignment device, aiding a surgeon in guiding and aligning interference screw 12 to a pre-tapped/pre-drilled implantation site.

Also, a portion of the head can be beveled to accommodate grafts (e.g. tendons or other types of grafts) as they are drawn over the angled face of the head and secured. This bevel can be further grooved longitudinally to facilitate grouping of ends of the grafts together and preventing migration of the grafts during placement of a washer.

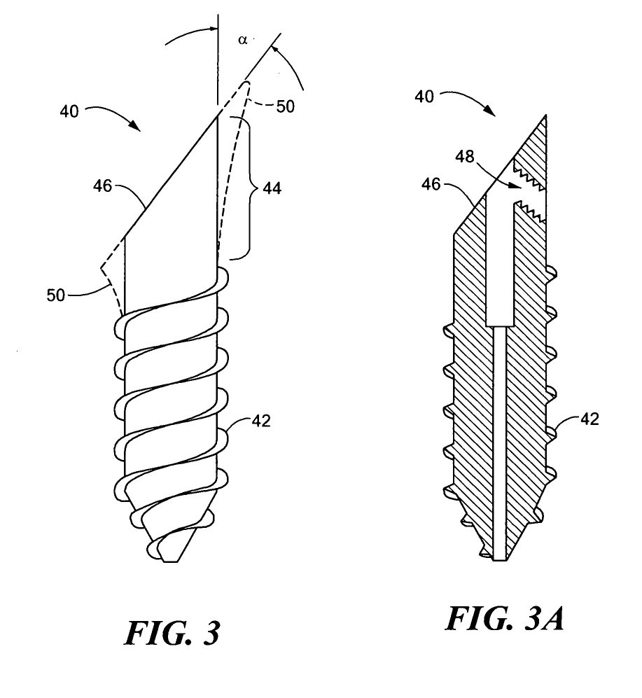

Referring now to FIGS. 3 and 3A in which like elements are provided having like reference designations throughout the several views, an interference screw 40, includes threads 42 which extend along a portion of an external surface thereof. A head end 44 of the interference screw does not include threads (i.e. the head end 44 of the interference screw 40 corresponds to an unthreaded portion of the interference screw 40). The head end 44 of the screw 40 is provided having a face 46 which intersects a central longitudinal axis of the screw 40 at an angle α. The angle α is selected based upon the location at which the screw will be inserted which in turn is based upon the particular application for which the screw is being used. For example, in the case wherein the graft anchor is being used to provide tibial fixation of a graft in an anterior cruciate ligament (ACL) knee reconstruction, the angle α is selected to be approximately forty-five degrees. With the face 46 at angle of forty-five degrees, once the screw 40 is inserted, into a tibial tunnel (made for the purpose of connecting graft ligaments between the tibia and the femur as is generally known), the head end 44 of the screw 40 extends a relatively small amount above the surface of the bone into which the screw is inserted. Thus, with a properly selected angle α, the face 46 of the screw is substantially flush with the surface of the bone into which the screw 40 is inserted.

Other applications (i.e. other than tibial fixation in ACL knee reconstruction) may benefit from an angle other than forty-five degrees. The angle may thus be selected as any angle between about 15 degrees to about 90 degrees (or −15 degrees to −90 degrees) as called for in any particular surgical application.

A first passage 46 (FIG. 3A) extends from a first end of the screw to a second end of the screw 40. The first passage has a first portion having a first diameter and a second portion having a second different diameter. A second threaded passage 48 (FIG. 3A) extends through a sidewall of the screw 40.

A central longitudinal axis 26 (FIG. 2) of the second passage 24 is provided at an angle which is perpendicular to the angle of the surface 22.

In an alternate embodiment, the screw 40 is provided having a flared portion 50 in the head region of the screw. The head region can be slightly flared in diameter, for example, for the purpose of taking up a greater volume within a hole provided in an outer cortex of a bone in which the interference screw is disposed.

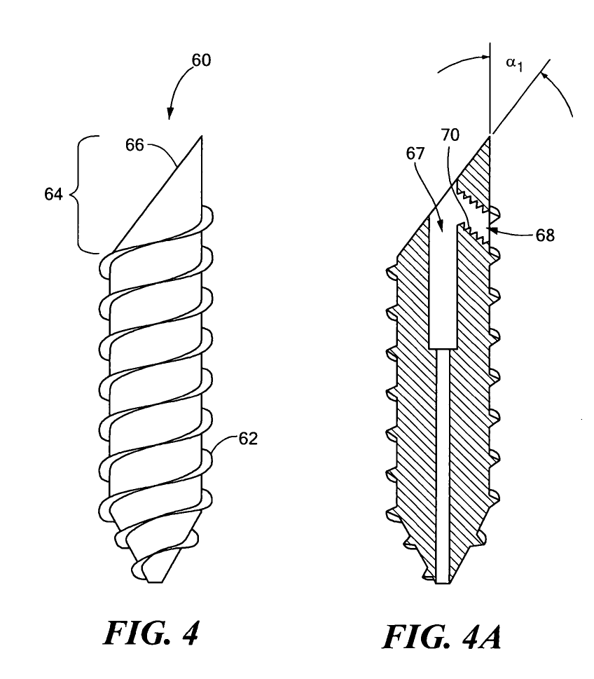

Referring now to FIGS. 4 and 4A in which like elements are provided having like reference designations throughout the several views, an interference screw 60, includes threads 62 which extend along the entire external surface thereof. A head end 64 of the interference screw is provided having a face 66 which intersects a central longitudinal axis of the screw 60 at an angle α. The angle α is selected based upon the location at which the screw will be inserted which in turn is based upon the particular application for which the screw 60 is being used. For example, in the case wherein the graft anchor is being used to provide tibial fixation of a graft in an anterior cruciate ligament (ACL) knee reconstruction, the angle α is selected to be approximately forty-five degrees. With the face 66 at angle of forty-five degrees, once the screw 60 is inserted, into a tibial tunnel (made for the purpose of connecting graft ligaments between the tibia and the femur as is generally known), the head end 64 of the screw 60 extends a relatively small amount above the surface of the bone into which the screw is inserted. Thus, with a properly selected angle α, the face 66 of the screw is substantially flush with the surface of the bone into which the screw 60 is inserted.

A first passage 67 (FIG. 4A) extends from a first end of the screw to a second end of the screw 60. The first passage has a first portion having a first diameter and a second portion having a second different diameter. A second passage 68 (FIG. 4A) intersects the first passage and extends through a sidewall of the screw 60 and includes grooves or pawls 70 selected to mate with corresponding grooves provided in a cross member (e.g. cross member 28 described above in conjunction with FIG. 1. The cross member is provided having corresponding grooves such that the cross member and grooved passage 68 form a pawl and ratchet structure.

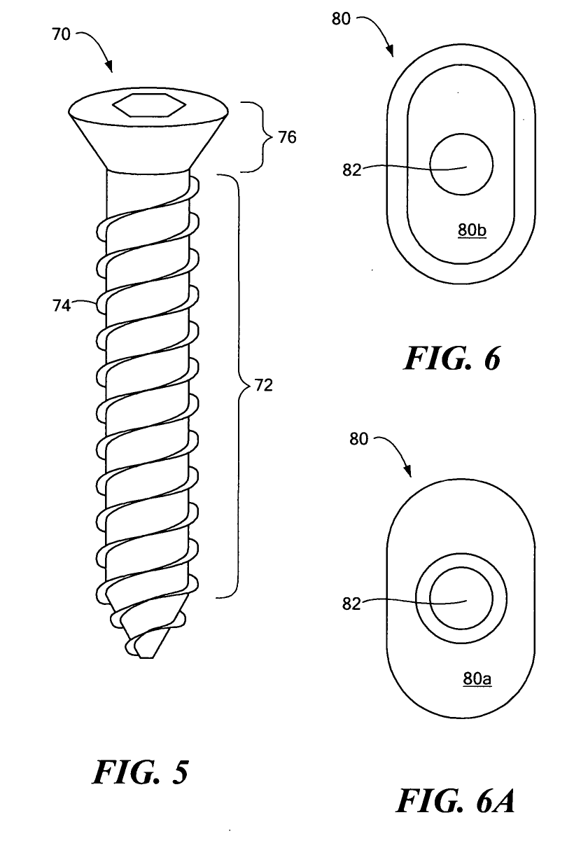

Referring now to FIG. 5, a cross member 70 is here provided as a cross screw having a body portion 72 with threads 74 thereon and a head end 76. The cross screw 70 may be adapted to mate with threads in a passage of an interference screw (e.g. one of the second passages 26, 48, 68 of the respective interference screws 12, 40, 60 described above in conjunction with FIGS. 1 and 2-4A).

In alternate embodiments, the head 76 of cross member 70 may include an eyelet of sufficient size to receive one or more sutures. The eyelet may be of any suitable size to accept any suture material or may come in a range of sizes specific to different suture types. In still further alternative embodiments, the head 76 may include a plurality of eyelets to enable one or more sutures to pass through two or more such eyelets. With these embodiments, when the cross member is inserted into the interference screw, the device can function as a suture anchor rather than as a graft or tendon anchor.

Referring now to FIGS. 6 and 6A, in which like elements are provided having like reference designations, a washer 80 includes a top surface 80a and a bottom surface 80b, with a tapered hole 82 therethrough. The hole is selected to have a size and shape which allows a cross member to be disposed therethrough. The washer 80 can be provided having an oval shape, a round shape or any other geometric shape including square, rectangular, hexagonal octagonal or even an irregular shape.

Referring now to FIGS. 7 and 7A, the insertion of a graft anchor used to enhance tibial fixation in an ACL knee rReconstruction is shown. A graft anchor, whch may be similar to the graft anchor 10 decsribed above in conjunction with FIG. 1, comprises a threaded interference screw 90, a washer 92 and a cross screw 94. After drilling or otherwsie providing a tunnel in the tibia 91 (i.e. a pre-drilled hole in the tibia) an ACL is reconstructed by placing a graft ligament 96 comprising strands 96a-96d in the pre-drilled hole. The interference screw 90 is placed along side strands 96a-96d of the graft liagament 96 in the pre-drilled hole. The interference screw is advanced into the hole until the screw head is not prominent above the tibia bone. One end of the graft ligament strands are coupled to the femur as is generally known (I should be appreciated that in FIG. 7, only portions of the graft end leading to the fenur are shown and that the portion of the graft ends leading to the femur are often looped). The other end of the graft ligament strands exiting the bone at the head end of the interference screw are drawn over the angled face 98 of the interference screw head. The cross screw 94 is advanced into a corresponding threaded hole in the interference screw. The cross screw advances the washer 92 such that a surface of the washer compresses the ends of the graft ligaments against the angled face 98 of the interference screw. The tibial anchor point is generally the weakest point in the reconstruction and the graft anchor device of the present invention improves the strength with which grafts are held at this point.

The combination of the cross screw, washer and angled face of the interference screw provides clamp-like function to hold the grafts in place. Both the angled face of the interference screw and/or the under surface of the washer may be provided having a textured surface so as to improved the grip with which the washer secures the graft strands to the face of the interference screw. The surfaces may be textured by providing grooves, notches, or other voids in the surfaces of the washer and/or angled face of the interference screw. Also, the mating surfaces of the interference screw and the washer may be shaped to improve the force with which the graft strands are secured therebetween (e.g. the angled surface of the screw may be shaped to improve mating with the surface of the washer).

It should be appreciated that in the case in which the washer is provided having an oval shape, the washer can be aligned with the major axis either horizontal or vertical with respect to major axis of the angled face with a horizontal position being preferred.

It should be further appreciated that the tibial anchor of the present invention can be used to anchor either a patellar tendon (bone-tendon-bone) or soft tissue graft. It should also be appreciated that the interference screws may be advantageously made from any suitable bio-compatible material, for example, titanium alloy, stainless steel, class six implant grade plastic, and the like, or any other bio-compatible material which exhibits adequate pullout strength and has sufficiently low brittleness to avoid breakage during long term usage of the device in suture. Alternatively, still if in a particular application an interference screw is used in an application that does not require a relatively long useful life of the screw, the interference screw may be made from a suitable bio-absorbable material, including but no limited to, polylactic, polyoxalic or polyglycolic acids.

Elements of different embodiments described herein may be combined to form other embodiments not specifically set forth above. Other embodiments not specifically described herein are also within the scope of the following claims.

What is claimed is:

1. A graft anchor comprising:
an interference screw having a first end, a second end, a sidewall having an outer surface, a first passage extending from the first end to the second end and a second passage having a first end which opens into at least a portion of the first passage and a second end which extends into at least a portion of the sidewall.

2. The anchor of claim 1 wherein the first end of the second passage terminates in an aperture which opens into at least a portion of the first passage.

3. The anchor of claim 1 wherein the second passage extends through the sidewall of the interference screw such that the second end of the second passage terminates in an aperture exposed on the outer surface of the interference screw.

4. The anchor of claim 1 wherein:
the first end of the second passage terminates in an aperture which opens into at least a portion of the first passage; and
the second end of the second passage terminates in an aperture exposed on the outer surface of the interference screw.

5. The anchor of claim 1 wherein at least a portion of the second passage has at least one thread.

6. The anchor of claim 1 wherein at least a portion of the second passage has at least one of: a groove, a tooth, a notch, an opening, or a hook.

7. The anchor of claim 1 wherein at least a portion of the second passage has at least one of a pawl or a ratchet.

8. The anchor of claim 1 wherein the second passage intersects the first passage and the second end of the second passage terminates in an aperture exposed on the outer surface of the interference screw.

9. The anchor of claim 1 further comprising a cross member disposed in the second passage of said interference screw.

10. The anchor of claim 1 wherein a first end of the cross member is disposed in the second end of the second passage and a second end of said cross member is disposed in the first end of the second passage and said anchor further comprises a washer disposed between the second end of the cross member and the first end of the second passage.

11. The anchor of claim 10 wherein the washer is provided as an integral part of the cross member

12. The anchor of claim 10 wherein the washer is provided having one of an oval shape, a round shape, a rectangular shape, a square shape or an irregular shape.

13. The anchor of claim 10 wherein a first surface of said washer is textured.

14. The anchor of claim 10 where the textured surface of said washer is provided by providing the first surface of said washer having at least one of bevels, grooves or notches.

15. The anchor of claim 7 wherein the cross member is provided as one of a screw, a pawl, a bolt, a dowel a pin or a rivet.

16. The anchor of claim 9 wherein the cross member is provided as a screw having at least one thread and the second passage has at least one thread adapted to mate with the at least one thread of the cross member.

17. The anchor of claim 1 wherein the first end of said interference screw is provided having an angled face.

18. The anchor of claim 1 wherein the angled face of the first end of said interference screw is provided having a textured surface.

19. The anchor of claim 18 where the textured surface is provided on the angled face of said interference screw by providing the angled face having at least one of bevels, grooves or notches

20. The anchor of claim 1 wherein the interference screw is provided having a first threaded portion and a second unthreaded portion.

21. The anchor of claim 1 wherein the interference screw is provided having one of: a pyramidal shape; a bullet shape; a barrel shape; a cone shape; a longitudinal cross-sectional shape corresponding to a wedge shape; or a barrel shape having a portion which is cone shaped.

22. A graft anchor comprising:
an interference screw having a head end and a distal end with a first passage extending from the head end to the distal end and a second passage provided in the head end; and
a cross member having a size and shape adapted to fit within the second passage.

23. The anchor of claim 22 wherein said interference screw is adapted to follow a guide wire.

24. The anchor of claim 22 wherein the outer surface of said interference screw is not threaded.

24. The anchor of claim 22 wherein the outer surface of said interference screw has a threaded portion and a non-threaded portion over the head end of the interference screw.

25. The anchor of claim 22 wherein the head of the interference screw is flared.

26. The anchor of claim 22 wherein the head end of the interference screw is provided having an angle such that it allows the interference screw head to be less prominent when inserted into a bone at oblique angle.

27. The anchor of claim 22 wherein the edges of the head end of the interference screw are beveled.

28. The anchor of claim 27 wherein the bevel is provided having grooves formed therein.

29. The anchor of claim 22 wherein the cross member is provided as cross screw and the second passage is provided as a threaded screw hole which accepts the cross screw.

30. The anchor of claim 22 wherein a central longitudinal axis of the threaded screw hole is provided at an angle which is 90 degrees to the angle of the face.

31. The anchor of claim 22 further comprising a washer and wherein the head end of the interference screw is provided having an angled face and wherein the washer is disposed between the angled face of the interference screw and a surface of the cross member.

32. The anchor of claim 31 wherein the washer is provided having a shape which is one of circular, elliptical, square or rectangular.

33. The anchor of claim 32 wherein a surface of the washer is textured.

34. The anchor of claim 32 wherein the angled face of the interference screw is textured.

35. A method for inserting a graft anchor comprising:
a) providing a hole in a bone;
b) inserting a graft strands through the hole;
c) inserting an interference screw into the hole such that the graft strands emerging from the hole at a head end of the interference screw;
d) drawing the graft strands over the head end of the interference screw; and
e) placing a cross member into a passage in the interference screw to secure the grafts against a surface of the interference screw.

36. The method of claim 35 wherein inserting the interference screw into the hole comprises advancing the interference screw into the hole until a head end of the interference screw is not prominent.

37. The method of claim 35 wherein the head end of the interference screw is angled and inserting the interference screw in the hole comprises advancing the interference screw into the hole until the angled head end of the interference screw is substantially flush with a surface of the bone.

38. The method of claim 35 wherein the cross member is a cross screw and the passage in the interference screw corresponds to a threaded hole and placing the cross member into the passage comprises placing the cross screw into the threaded hole.

39. The method of claim 38 further comprising placing a washer over the angled face of the head end of the interference screw and wherein placing the cross screw into the threaded hole comprises placing the cross screw into the threaded hole to advance the washer against the angled surface so as to compress the graft strands against the angled face of the interference screw.
